(19) United States
(12) Patent Application Publication
(10) Pub. No.: US 2013/0072436 A1
(43) Pub. Date: Mar. 21, 2013

(54) PROCESS FOR MANUFACTURING COLLAGEN AND TRACE ELEMENTS FROM SEA WATER

(71) Applicants: Yongsheng Qiu, Bangkok (TH); Hsin-Ken Wang, Phingjeon (TH)

(72) Inventors: Yongsheng Qiu, Bangkok (TH); Hsin-Ken Wang, Phingjeon (TH)

(73) Assignee: SEAWAGEN CO., LTD., Bangkok (TH)

(21) Appl. No.: 13/638,071

(22) PCT Filed: Jul. 30, 2010
(86) PCT No.: PCT/TH10/00026

§ 371 Date: Sep. 28, 2012

(30) Foreign Application Priority Data
Jan. 11, 2010 (TH) 1001000037

(51) Int. Cl. A61K 38/39

(52) U.S. Cl. CPC A61K 38/39
USPC 514/17.2

(57) ABSTRACT

The process for manufacturing collagen and trace elements from sea water consists of various steps which are raw material preparation, resting of raw material, preliminary filtration to remove heavy metals, deodorize and purity remaining impurities, spraying of water, separation of heavy metals, collagen and trace elements from water and separation of collagen and trace elements. The process according to this invention, apart from sea water, can be applied to other type of salt water by adjusting the salinity and density of the water to an optimal value. The process according to this invention yields clean and purified collagen which is easily absorbed by human body and contains numerous types of trace elements.

### CROSS-REFERENCE TO RELATED APPLICATIONS

This application is the U.S. national stage of International (PCT) Patent Application No. PCT/TH2010/000026, filed Jul. 30, 2010, which claims priority to Thai Patent Application No. 1001000037, filed Jan. 11, 2010. The entire disclosures of these two applications are incorporated herein by reference in their entireties

### FIELD OF THE INVENTION

Food sciences and technology related to process for manufacturing collagen and trace elements from sea water

### BACKGROUND OF THE INVENTION

Collagen is the composition of tissues between cells in the human body. Currently, collagen has been used in cosmetic, nutraceutical, pharmaceutical and medical products. The benefits of collagen are, for instance, wound healing, adjusting equilibrium of epidermis, retaining moisture of skin and improving flexibility of subcutaneous tissues.

From the scientific discovery, collagen (33% in form of egg white) is in human body's structure. After 25 years, collagen within the body decreases at the rate of approximately 1.5%.

Trace elements or micro elements are present in the human body less than 0.01% of body weight. At present, 80 trace elements in human body have been found. The significance of trace elements to human body includes;
1. Protect the normal function of human body
2. Control circulation in cell and systems
3. Improve immune.
4. Enhance cell potentials

In general, the processes of manufacturing collagen include chemical separation and separation by fermentation and raw materials are derived from terrestrial and aquatic animals as well as selected plants. In case of terrestrial animals i.e. cow, pig, goat and chicken, the starting materials are skin, bone and tendons. In case of aquatic animals i.e. fish and shrimp, the starting materials are shell, skin and bone whereas, in case of plant, the starting material is bark.

The said general processes of manufacturing collagen yield acidic collagen with only a few types of trace elements which cannot be adjusted for application in human completely.

### SUMMARY OF THE INVENTION

This invention is related to the process for manufacturing collagen and trace elements from sea water or salt water. The process includes the steps of raw material preparation; resting of raw material; preliminary filtration to remove heavy metals, deodorize and purify remaining impurities; spraying of water, heavy metals, collagen and trace elements separation from water and separation of collagen and trace elements.

The aim of this invention is to provide the process for manufacturing collagen and trace elements from sea water or salt water by using sea water or salt water as raw material. This process, therefore, yields the purified collagen which is easy to be absorbed by human body and contains numerous types of trace elements.

Collagen extracted from sea water has the smallest size of molecular weight (3000) when compared to collagens derived from animals (terrestrial and aquatic).

Collagen containing high amount of trace elements is essential for human body. The coordinating angle of human body is close to the coordinating angle of sea water which is between 165-180° whereas the coordinating angle of freshwater is 130°. This makes the collagen with high amount of trace elements easy to be absorbed.

Therefore the extraction of collagen and trace elements from sea water is not only important for the pharmaceutical and cosmetic applications but also for the human well-being and disease prevention.

The other objectives of this invention can be understood by the person skilled in the art according to the detail description of this invention.

### DETAILED DESCRIPTION OF THE INVENTION

The following details describe the process for manufacturing collagen and trace elements from sea water in order to further explain the details of this invention.

The process for manufacturing collagen and trace elements from sea water according to this invention includes the steps;

(1) Raw Material Preparation:

In this step, the sea water is transferred to the container with air circulation in order to ventilate the air, deodorize, purify impurities and remove heavy metals from the said sea water.

Raw materials according to this invention, besides the said sea water, another type of salt water may also be used as the raw materials, for example, saline water (prepared from salts). In order to optimize the process, when salt is used to prepare saline for the process according to this invention, the salinity and density should be adjusted to the optimal values.

Moreover, the containers can be selected from but not limited to bucket, well or any other containers.

(2) Resting of Raw Material

In this step, the raw material is rested for a certain period of time according to its type.
Seawater is rested for 1-2 days
Salt water is rested for 2-3 days

(3) Preliminary Filtration to Remove Heavy Metals, Deodorize and Purify Remaining Impurities

This step involves re-filtering the sea water or salt water resulting in multiple transferring of raw material in order to deodorize, purify remaining impurities and remove heavy metals i.e. mercury and lead from the water and to aerate and foam in order to deodorize and purify the remaining impurities. This step yields clean and odorless water.

The process in this step is performed by passing the sea water or salt water into the filtering machine repeatedly.

(4) Spraying of Water

The water obtained from (3) will be sprayed within the tube under the controlled temperature and flow rate and re-filtered with the filtering machine before storing in a resting container.

The said resting container includes a resting bucket or any other resting containers.

(5) Separation of Heavy Metals, Collagen and Trace Elements from Water

The water obtained from (4) is passed through the separation machine by circulating water within the separation machine several times in order to remove heavy metals. The heavy metals is then collected whereas collagen and trace elements are still binding and suspending in water.

The separation machine is based on the magnetic field and able to separate heavy metals, collagen and trace elements. After heavy metals, collagen and trace elements separation, the salinity of water is reduced and the appearance of water is milky.

(6) Separation of collagen and trace elements

In this step, the water with reduced salinity and milky appearance obtained from (5) will be precipitated in order to separate collagen and trace elements or the said water can be filtered with fine filter cloth in order to remove collagen and trace elements. The separation can be done with any other methods. The collagen and trace elements obtained from this step is grayish-white gel-like liquid.

To obtain a better result, the step of raw material preparation should be conducted under controlled pressure and light of the sea water so that it optimally helps to generate collagen and trace elements in a sufficiently large amount.

In addition, in order to obtain a better result, the step of preliminary filtration to remove heavy metals, remaining impurities and deodorize should be conducted by increasing pressure and oxygen in order to reduce the size of the elements molecules, making it easier for the microbes to absorb these elements and encouraging microbes to grow faster and live longer.

The process for manufacturing collagen and trace elements from sea water according to the invention yields the purified collagen which is easily absorbed by human body and contains many types of trace elements that are essential for human body.

The process for manufacturing collagen and trace elements from sea water according to this invention may be modified and altered within the scope of the invention.

1. The process for manufacturing collagen and trace elements consist comprising
Raw material preparation by storing sea water in a container, controlling the pressure and light of the sea water so that it is suitable, and making the air circulate for the purpose of preliminary air ventilation, deodorization, removing impurities and heavy metals from the said sea water;
Preliminary filtration to remove heavy metals, remaining impurities and deodorize to remove heavy metals and remaining impurities, increasing pressure and oxygen and foaming of water in order to deodorize which yields the clean and odorless water;
Spraying of water wherein clean and odorless water is sprayed within the tube under the controlled temperature and flow rate, and re-filtered with the filtering machine before storing in a resting container;
Separation of heavy metals, collagen and trace elements from water by passing the obtained water through separation machine several times in order to additionally remove heavy metals; and
Separation of collagen and trace elements.

2. The process for manufacturing collagen and trace elements according to claim 1 wherein the additional step is the resting of raw material following the raw material preparation.

3. The process for manufacturing collagen and trace elements according to claim 1 wherein the raw material includes salt water, preferably the said salt water is saline water, and its salinity and density are adjusted to the optimal values.

4. (canceled)

5. The process for manufacturing collagen and trace elements according to claim 1 wherein the container is a bucket or a well, wherein the resting container includes a resting bucket.

6. The process for manufacturing collagen and trace elements according to claim 2 wherein the resting of raw material is performed by resting the sea water or salt water in a container for a certain period of time, preferably the resting of sea water is performed in the container for 1-2 days while the resting of salt water is for 2-3 days.

7-8. (canceled)

9. The process for manufacturing collagen and trace elements according to claim 1 wherein the filtering of sea water in order to remove heavy metals is performed by passing the water into the filtering machine several times.

10. The process for manufacturing collagen and trace elements according to claim 1 wherein the said separation machine is based on the magnetic field and able to separate heavy metals, collagen and trace elements.

11. The process for manufacturing collagen and trace elements according to claim 1 in which involves the step of heavy metals, collagen and trace elements separation, the heavy metals is then separated whereas collagen and trace elements are still binding and suspending in water.

12. The process for manufacturing collagen and trace elements according to claim 1 in which involves the step of heavy metals, collagen and trace elements separation, after heavy metals, collagen and trace elements separation, the salinity of water is reduced and the appearance of water is milky.

13. The process for manufacturing collagen and trace elements according to claim 1 wherein the separation of collagen and trace elements is performed by precipitation of the water with reduced salinity and milky appearance.

14. The process for manufacturing collagen and trace elements according to claim 1 wherein the separation of collagen and trace elements is performed by filtration of the water with reduced salinity and milky appearance, preferably with fine filter cloth.

15. (canceled)

16. Collagen and trace elements obtained from the processes according to claim 1.

17. Collagen and trace elements obtained from the processes according to claim 3.

18. Collagen and trace elements obtained from the processes according to claim 10.

19. Collagen and trace elements obtained from the processes according to claim 13.

20. Collagen and trace elements obtained from the processes according to claim 14.
